(19) United States
(12) Patent Application Publication
(10) Pub. No.: US 2015/0250740 A1
(43) Pub. Date: Sep. 10, 2015

(54) BIOADHESIVE PATCH

(71) Applicant: Swedish Pharma AB, Stockholm (SE)

(72) Inventors: Paul A. MCCARRON, Newtownabbey (GB); Ryan F. DONELLY, Co Down (GB); David WOOLFSON, Belfast (GB)

(21) Appl. No.: 14/690,666

(22) Filed: Apr. 20, 2015

Related U.S. Application Data
(63) Continuation of application No. 13/132,900, filed on Aug. 15, 2011.

(30) Foreign Application Priority Data
Dec. 4, 2008 (SE) 0850117-3

(51) Int. Cl. A61K 9/70; A61K 31/465; B32B 27/08; B32B 37/06; B32B 37/10; B32B 7/12; A61K 31/197; A61K 45/06

(52) U.S. Cl. CPC A61K 9/7053; A61K 31/197; A61K 31/465; A61K 45/06; B32B 37/06; B32B 37/10; B32B 7/12; B32B 27/08; B32B 2309/02; B32B 2535/00; B32B 2327/06; B32B 2333/04; B32B 2371/00; B32B 2556/00

(57) ABSTRACT

A moist, layered bioadhesive patch includes one or more polymers. A method of producing a monolayered film and a method of drying said film is provided. Additionally, there is provided a method of producing bioadhesive, layered patches by combining layers of the monolayered film to obtain a desired thickness of the patch. Patches according to the invention may be used as such, or for delivering pharmaceutically active compounds, such as in a drug delivery system.

### FIELD OF INVENTION

The present invention relates to a moist, layered bioadhesive patch comprising one or more polymers. Moreover, the invention relates to a method of producing a monolayered film and a method of drying said film. Additionally, the invention relates to methods of producing bioadhesive, layered patches by combining layers of the monolayered film to obtain a desired thickness of the patch. Patches according to the invention may be used as such, or for delivering pharmaceutically active compounds, such as in a drug delivery system.

### BACKGROUND

Moisture compromises the adhesion of pressure sensitive adhesive-based (PSA) devices (Moon et al., 2002), meaning that they may not stay in place long enough to be clinically effective, especially in wet environments, such as the mouth or the lower female reproductive tract.

In contrast bioadhesive drug delivery systems adhere strongly to biological substrates in wet environments. As a result, they facilitate prolonged residence times and concomitant increases in drug absorption at a number of sites in the human body. These include the eye, the nose, the vagina and the gastrointestinal tract.

Bioadhesive drug delivery systems have been formulated as powders, compacts, sprays and semi-solids, as well as patches. For example, polymeric powders have been used for drug delivery to the nasal mucosa (Nagai and Konishi, 1984), compacts and microspheres have been developed for use in the oral cavity (Ponchel et al., 1987; Kockisch et al., 2003) and patches, consisting of a bioadhesive layer and a non-adhesive backing layer, have been used for topical drug delivery to the skin (McCafferty et al., 2000; Donnelly et al., 2006; McCarron et al., 2006).

Proprietary bioadhesive products include compacts (eg Corlan® pellets containing hydrocortisone) and creams (eg Clindesse™ vaginal cream containing clindamycin). However, no bioadhesive patch system is currently marketed. This lack of proprietary bioadhesive patches is largely due to the fact that such systems are exclusively water-based, meaning drying is difficult. Removal of water from a drying system requires much more time and energy than the removal of volatile organic solvents used in the casting of pressure sensitive adhesive patches. In addition, during protracted drying periods, volatile drugs can evaporate and drugs incorporated at high loadings can crash out of solution, thus reducing the concentration drive for drug diffusion into the skin, impairing adherence and spoiling the aesthetic appearance of the formed patch.

Currently all patch-based drug delivery systems available commercially are PSA-based. As a result, for example neoplastic and dysplastic lesions on the lip, in the mouth, on the vulva or in the vagina, which could be ideally treated using prolonged local patch-mediated drug delivery, are only ever treated in this manner in clinical trials.

### DESCRIPTION OF INVENTION

The present invention provides a layered patch, methods of producing the same from monolayered film, as well as a method of producing said monolayered film, by way of which the problems associated with currently available patches may be overcome. A drying method for the monolayered film is moreover provided. Use of the patch is also claimed.

According to a first aspect of the invention, there is provided a moist, layered bioadhesive patch as defined in the appended claims. The patch comprises one or more polymers chosen from a group comprising poly(methyl vinyl ether/maleic acid) and esters/amides thereof, poly(methyl vinyl ether/maleic anhydride), poly(acrylic acids) and esters/amides thereof, and chitosan and cellulose derivatives.

In one embodiment, the moist, layered bioadhesive patch further comprises at least one plasticizer chosen from a group comprising glycerol, propylene glycol, poly(ethylene glycol) and tripropylene glycol monomethyl ether (TPM).

Advantages of the moist patch of the invention is that it adheres strongly to humid or wet environments of the body, such as mucosa, and to skin. In addition to adhering strongly, the moist patch retains its position for long periods of time, thus enabling coverage of an area for an extended period of time.

The moist patch is suitable for treating e.g. sores of the mouth, such as cold sores. Moreover, the moist patch may be used for protecting organs after trauma, especially organs that are difficult to treat surgically. In this context, the moist patch is particularly useful for protecting the eyeball and internal organs, such as the liver, from leakage after trauma. The moist patch may hence be used as a bandage.

The component parts of the patch, i.e. the selected polymer(s), possibly in combination with biopolymer(s) such as any polysaccharide and/or cellulose derivative, make the moist patch flexible and easily handled clinically. Thereby, the moist patch is easily attached to a moist or wet surface.

By way of its flexibility, the moist, layered patch conforms to irregularly shaped body surfaces when in use, both internal and external body surfaces, including mucosa-lined body surfaces.

The term patch is used herein as a denomination for the moist, layered bioadhesive patch in a condition ready to be used, i.e. containing all layers desired. That is not to say, however, that the moist, layered patch as manufactured and/or supplied necessarily has a size suitable for its end use. The moist, layered patch may be easily cut to a desired size and shape, since this would not cause leakage of the pharmaceutically active compound(s) that may be contained therein.

The term film is used herein as a denomination for a monolayered film, from which the patch is produced. In one embodiment, the moist, layered bioadhesive patch according to the invention comprises at least two film layers, such as 3, 4, 5, 6, 7, 8, 9, 10, 11, 12, 13, 14, 15, 16, 17, 18, 19 or 20 film layers. The thickness of the film layers may be adjusted to the total number of film layers desired, such that the resulting moist, layered bioadhesive patch gets the desired thickness.

The moist, layered bioadhesive patch comprises according to one embodiment of the invention in one or more film layers independently of one another at least one pharmaceutically active compound. Since the moist patch may retain its position for long periods of time, it enables pharmacological treatment regimes requiring extended treatment periods. Moreover, the non-leakage of pharmaceutically active compound(s) when the moist patch is cut further facilitates pharmacological treatment.

The pharmaceutically active compound(s) may be chosen from a group comprising nicotine, 5-ALA (5-aminolevulinic acid) and derivatives thereof, antibiotics, parasympatholytics, cholinergics, neuroleptics, antidepressants, antihypertensives, photosensitisers, photosensitiser precursors, sympathomimetics, sympatholytics and anti-sympathotonics, antiolytics, local anaesthetics, central analgesics, antirheumatics, coronary therapeutics, hormones, antihistamines, prostaglandin derivatives, vitamins, nutrients, cytostatics and locally active anti-cancer compounds such as, but not limited to, Rose Bengal as well as systemically active anti-cancer compounds. The pharmaceutically active compound(s) may be in the form of salt(s). Active compounds may be delivered to the eye, including the cornea, sclera and other parts, such as sensitizers for treatment of infection, tumours etc. Moreover, the moist patch may comprise additives or auxiliaries such as permeation enhancers, stabilizers, fillers, tackifiers, absorption promoters etc. The patch may also be used as a bandage to protect tissue from mechanical irritation e.g. sores on mucosal and epithelial surfaces and as a bandage to protect organs from leaking vital contents such as vitreous humour after trauma to the eyeball. With a resorbable backing the patch may also be used to seal bleeding internal organs. By choosing suitable backings the patch, with or without active compounds, can be used to diminish mechanical irritation and pressure on tissues exposed to a moist, wet and hash environment.

By way of its layered structure, the moist patch may have different pharmaceutically active compounds in different layers, so as to enable a combination therapy with one and the same moist patch. Alternatively, several or all layers may contain the same pharmaceutically active compound or the same mixture of pharmaceutically active compounds.

Providing a film layer located close to e.g. the mucosa when used with e.g. an anaesthetic, the moist patch may be used as a delivery system for administering a local anaesthetic to relieve pain. The moist patch may also find its use for administration of local anaesthetics e.g. prior to surgical procedures carried out under local anaesthesia. By providing a film layer located close to e.g. the mucosa when used with e.g. a permeation enhancer, the efficacy of uptake to the body of pharmaceutically active compound contained in the same or different film layer(s) as the permeation enhancer may be improved.

In one embodiment of the invention, the moist, layered bioadhesive patch, the pharmaceutically active compound added is 5-aminolevulinic acid, or a derivative or salt thereof, and is present in the first and/or further film layers in an amount in the range of 1-50 mg cm⁻².

In another embodiment of the invention, the moist, layered bioadhesive patch, the pharmaceutically active compound added is nicotine, and is present in the first and/or further film layers in an amount in the range of 1-30 mg cm⁻².

In one embodiment of the invention, each film layer has a thickness of 1 μm to 500 μm, preferably 25 μm to 75 μm and most preferably approximately 50 μm. The combined layers to form the patch preferably provide a patch having a thickness in the range of 2 μm to 1000 μm, with a preferred thickness being in the range of 0.5 mm to 5 mm and the most preferred thickness being approximately 1 mm.

In one embodiment of the invention, each film layer of the moist, layered bioadhesive patch exhibits a tensile strength greater than 1.0×10⁻⁸N cm⁻² and a residual tackiness, such that detachment of two layers of the same material requires a force of removal>1.0 N cm⁻².

In another embodiment, the invention provides a moist, layered bioadhesive patch further comprising a backing layer. It is preferred that the backing layer comprises a flexible, water-insoluble polymeric material, such as a film prepared from polyvinylchloride (PVC) emulsion or the like, such as a Plastisol® emulsion. The plasticizer used in Plastisol® is diethylphthalate but any similarly suitable plasticizer may be used, depending on the nature of the film comprising the backing layer.

A moist, bioadhesive patch being of such a small thickness as not to be easily handled may be provided with a backing layer to counteract otherwise suboptimal handling properties. A suitable thickness of the backing layer is easily chosen by the person skilled in the art.

In a further embodiment of the first aspect of the invention, the moist, layered bioadhesive patch the provided with a moisture impermeable polyester foil to protect the patch when not in use. The polyester foil is preferably easily removable.

According to a second aspect of the present invention, there is provided a method of producing a monolayered film as defined in the appended claims, said method comprising:
(a) providing an aqueous solution comprising at least one polymer chosen from a group comprising poly(methyl vinyl ether/maleic acid) and esters/amides thereof, poly(methyl vinyl ether/maleic anhydride) (PMVE/MA), poly(acrylic acids) and esters/amides thereof;
(b) spreading out or spraying a thin layer of the solution resulting from (a) to form a film;
(c) drying the film formed.

In one embodiment, the polymer is (PMVE/MA) and is present in an amount of 0.5% w/w to 50% w/w of the aqueous solution, preferably around 20% w/w of the aqueous solution.

Thin layer as defined herein is used as a term for a film layer having a thickness from 1 μm. The thickness of the film prepared may by the person skilled in the art be chosen to be commensurate with the desired thickness of the patch to be obtained and its use. The term “monolayered film” as used herein is serving elucidatory purposes only, since all films used herein are monolayered.

In one embodiment of the above, second aspect of the invention, the method of producing a monolayered film comprises addition of a pharmaceutically active compound to the aqueous solution of (a). Such a pharmaceutically active compound may be chosen from a group comprising nicotine, 5-ALA and derivatives thereof, antibiotics, parasympatholytics, cholinergics, neuroleptics, antidepressants, antihypertensives, photosensitisers, photosensitiser precursors, sympathomimetics, sympatholytics and antisympathotonics, antiolytics, local anaesthetics, central analgesics, antirheumatics, coronary therapeutics, hormones, antihistamines, prostaglandin derivatives, vitamins, nutrients, cytostatics, locally active anti-cancer compounds, systemically active anti-cancer compounds. Also included in the aqueous solution can be additives or auxiliaries such as permeation enhancers, stabilizers, fillers, tackifiers, absorption promoters etc.

Examples of suitable pharmaceutically active compound(s) include 5-aminolevulinic acid (or a derivative or salt thereof), which is a porphyrin precursor used in photodynamic therapy and must be incorporated at high loadings, and nicotine which is a volatile drug used in nicotine replacement products for smoking cessation therapy.

In yet an embodiment of the second aspect of the invention, the film formed is dried for a period of less than 30 minutes. The film formed needs to be moist to allow the subsequent adherence of two film layers to one another. If the first film is wet when applied to a second, moist film, the second film will be at least partially dissolved, with the resulting disadvantage of extended drying periods. If the first film is dry when applied to a second, moist film, the first film may not adhere to the second film. Similarly, if the patch dries out, it tends to fall apart. “Moist” is used herein as a synonym to the terms “dry to touch” or “touch dry”, whereby the film is still tacky and not absolutely dry and is resistant to viscous flow within a reasonable timeframe (eg <24 hours) and has a tensile strength greater than 1.0×10⁻⁸N cm⁻². Tackiness is defined herein as the film being sufficiently adhesive to bind to another layer of the same material, such that detachment of the two film layers requires a force of removal>1.0 N cm⁻². The film may be in touch dry condition after drying for a period of about 15 minutes.

In one embodiment of the second aspect of the invention, the aqueous solution of (a) further comprises at least one plasticizer chosen from a group comprising glycerol, propylene glycol, poly(ethylene glycols) and tripropylene glycol monomethyl ether (TPM). (TPM) may then be introduced in an amount of from 0.25 to 25% w/w of the aqueous solution, preferably around 10% w/w thereof.

In one embodiment, the aqueous solution further comprises a water-miscible co-solvent. This co-solvent may be ethanol and/or acetone and be present in an amount of 0.1% w/w to 80% w/w, respectively, of the aqueous solution. If the co-solvent is ethanol, it is preferably in the region of about 30% w/w of the aqueous solution. If the co-solvent is acetone, it is preferably present in the region of about 22% w/w of the aqueous solution.

There is provided a monolayered film as manufactured in accordance with the second aspect of the invention.

The film may be produced in sizes of approximately 5 cm by 3 cm. However, other sizes can be utilised if necessary or desired. If the films are to be used to form a transdermal patch, the patch once produced may be sealably packaged in a moisture impermeable polyester foil. The patch may be made to vary in size as required by the end user.

According to a third aspect of the invention, there is provided a method of making a moist, layered bioadhesive patch as defined in the appended claims, said method comprising
(i) providing a monolayered film produced in accordance with the second aspect of the invention
(ii) providing a backing layer
(iii) applying a first layer of the monolayered film on the backing layer
(iv) applying a second layer of the monolayered film on the first film layer
(v) pressing the film layers together until the film layers adhere
(vi) optionally repeating steps (iii)-(v) to build up the patch to a desired thickness.

There is provided a moist, layered bioadhesive patch manufactured in accordance with the third aspect of the invention.

A sequential in-line manufacturing arrangement as set out in FIG. 7 herein may provide an efficient production means of a moist, bioadhesive patch according to the invention.

According to a fourth aspect of the invention, there is provided a method of making a moist layered bioadhesive patch as defined in the appended claims, said method comprising
(i) providing the monolayered film produced in accordance with the second aspect of the invention
(ii) applying the monolayered film on a backing layer
(iii) folding the backing layer with the applied film, such that film surfaces confront
(iv) pressing the folded film layers together until the first and second film layers adhere
(v) removing at least a portion of the backing layer to expose part of the film,
(vi) optionally repeating steps (iii)-(v) to build up the patch to a desired thickness.

There is provided a moist, layered bioadhesive patch manufactured in accordance with the fourth aspect of the invention.

In one embodiment of the third and fourth aspects of the invention, respectively, a pharmaceutically active compound is present in at least one of the film layers. The pharmaceutically active compound is chosen from the group comprising compounds already disclosed hereinabove. Additionally, additives or auxiliaries may be included in at least one film layer.

In one embodiment of the third and fourth aspects of the invention, a support substrate of any suitable material on which to form the film may be provided, such as a glass substrate.

According to a fifth aspect of the invention, which is defined in the appended claims, there is provided use of the monolayered film as described herein in the manufacture of a moist, layered bioadhesive patch in accordance with the first aspect of the invention. The moist, layered bioadhesive patch may be a transdermal patch, a transmucosal patch or a topical patch. In one embodiment, the monolayered film is used in the manufacture of a drug delivery patch for delivery of pharmaceutically active compounds to mucosa-lined parts of the body.

The present invention describes a unique drying technique which allows manufacture of moist bioadhesive patches in a similar timeframe to that employed in the drying of PSA-based patches.

Accordingly, a sixth aspect of the invention, as defined in the appended claims, relates to a method of drying a film layer for use in the production of a monolayered film according to the second aspect of the invention, said method comprising an air drier for drying a monolayered film, wherein an airflow venturi having a housing and a plurality of fans located within at least one wall thereof and adapted such that the fans can draw in warm air having a temperature of between 5° C. and 150° C. and blow it over the film to be dried, said drier optionally containing within the housing a thermostatically controlled hot plate on which the film to be dried is placed. It is preferred, but not essential, that the hot plate is maintained at a temperature between 15° C. to 100° C., with the most preferred temperature between 20° C. and 60° C. and ideally around 20° C.

In one embodiment of the sixth aspect of the invention, the fan draws in warm air having a temperature range of 15° C. and 80° C., preferably a temperature in the range of 20° C. to 60° C., whereas the hot plate may be maintained at a temperature in the range of 15° C. to 100° C.

In another embodiment of the invention, the method of drying a film layer according to the sixth aspect of the invention, the method comprises placing a film layer to be dried in the above-described drier, blowing warm air over the film layer to be dried for a period in the region of 15 minutes or until the film layer is touch dry, which ever is shorter. The drying process preferably lasts no longer than 30 minutes.

In a seventh aspect of the invention, there is provided a method of drying the aforementioned monolayered film, the method comprising infrared lamp(s) and/or microwave generator(s) for heating the monolayered film, whereupon or during which heating period cold air is optionally blown over the film for the film not to be heat-damaged. The person skilled in the art realizes that cooling needs to be in parity with the heat-induced curing of the film, so that the film is not destroyed during curing.

The invention is further described below with reference to the following examples and tables.

### BRIEF DESCRIPTION OF THE DRAWINGS

The invention is also illustrated with reference to the accompanying figures in which:

FIGS. 1(A)-1(B) are diagrammatic representations of the methods used to prepare thin films from Plastisol® PVC emulsion (A) and aqueous blends of PMVE/MA, TPM (tripropylene glycol monomethyl ether) and ALA (Aminolevulinic Acid) (B).

FIG. 2 is a diagrammatic representation of the film dryer.

FIGS. 3(A)-3(D) are diagrammatic representations of the steps involved in the preparation of moist, bioadhesive patches containing ALA (Aminolevulinic Acid) by a multiple lamination (i.e. film-applying coating) method according to the invention using thin monolayered films. Thin, monolayered bioadhesive film containing ALA on PVC film attached to glass plate (A). Start of folding process, film divided into 3 cm×5 cm sections and sections folded onto the adjacent segment in a sequential fashion (B). Intermediate stage in the folding process (C). Completion of the folding process; adjacent sections folded on top of one another, bonded by the application of gentle pressure and the PVC backing peeled off (D).

FIGS. 4(A)-4(E) illustrates the typical: (A) melting endotherm observed for ALA (3.8 mg); (B) DSC trace for a cast monolayered film containing no ALA; (C) DSC trace for a layered patch containing no ALA; (D) DSC trace for a cast monolayered film containing 50 cm⁻² ALA; (E) DSC trace for a layered patch containing 50 cm⁻² ALA.

FIGS. 5(A)-5(D) illustrate the influence of ALA loading and preparation method on (A) adhesion of bioadhesive films to shaved neonate porcine skin (mean±S.D., n=5); (B) on distance to separation of bioadhesive films and shaved neonate porcine skin (mean±S.D., n=5); (C) on the break strengths of films prepared from aqueous, or aqueous alcoholic, blends containing 20% w/w PMVE/MA and 10% TPM (mean±S.D., n=5); (D) on the percentage elongations at break of films prepared from aqueous, or aqueous alcoholic, blends containing 20% w/w PMVE/MA and 10% TPM (mean±S.D., n=5).

FIGS. 6(A)-(C) illustrate the cumulative release of ALA from a bioadhesive patch prepared by multiple lamination and casting methods across Cuprophan® membranes in which (A) both formulations were tailored to deliver 19 mg ALA cm⁻². Results are plotted as mean values±S.D. (n=3); (B) both formulations were tailored to deliver 38 mg ALA cm⁻² and the results are plotted as mean values±S.D. (n=3); (C) both formulations were tailored to deliver 50 mg ALA cm⁻² and the results are plotted as mean values±S.D. (n=3).

FIG. 7 illustrates an example of a sequential in-line manufacturing arrangement to provide the patch of the present invention.

FIG. 8 illustrates an example of a parallel manufacturing arrangement with continuous film use at each layer pre-stage to provide the patch of the present invention.

The following examples now set out to describe the invention further.

Two model drugs have been used to illustrate the process. The first of these, 5-aminolevulinic acid (or salt thereof) (ALA), is a porphyrin precursor used in photodynamic therapy (PDT), and must be incorporated at high loadings. This is due to the fact that large topical doses are required clinically because of poor skin penetration (Donnelly et al., 2005). High drug loadings provide a greater concentration drive for diffusion, but also tend to cause precipitation of crystals during protracted drying.

The second drug is nicotine, which is used in various nicotine replacement products for smoking cessation therapy (BNF 52). This drug is reasonably volatile (bp 247° C., Merck Index 14^{th} Edition) and is likely to evaporate during prolonged drying periods.

Nicotine and tripropyleneglycol methyl ether (Dowanol™ TPM) were purchased from Sigma Aldrich, Dorset, UK. Gantrez® AN-139, a copolymer of methyl vinyl ether and maleic anhydride (PMVE/MA), was provided by ISP Co. Ltd, Guildford, UK. Plastisol® medical grade poly(vinyl chloride) (PVC) emulsion, containing diethylphthalate as plasticiser, was provided by BASF Coatings Ltd., Clwyd, UK. All other chemicals used were of analytical reagent quality. Poly(ester) film, one-side siliconised, release liner (FL2000™ PET 75μ 1S) was purchased from Rexam Release B.V., Apeldoorn, The Netherlands. Moisture-impermeable, heat-sealable poly(ester) foils were purchased from Transparent Film Products Ltd., Newtownards, N. Ireland. Aminolevulinic acid hydrochloride salt (ALA) was purchased from Crawford Pharmaceuticals, Milton Keynes, UK.

### EXAMPLE 1

### Drug Incorporated at High Loading

### Methods and Materials

The casting method is a method well known in the prior art and is used by way of comparison with the embodiments of the present invention

### Preparation of Bioadhesive Patches Containing ALA by Casting

Aqueous polymer blends were prepared, as described previously (Donnelly et al., 2006), using the required weight of poly(methylvinylether-co-maleic anhydride) (PMVE/MA), which was added to ice-cooled water and stirred vigorously. The mixture was then heated and maintained between 95° C. and 100° C. until a clear solution was formed. Upon cooling, the required amount of tripropylene glycol methyl ether (TPM) was added and the casting blend adjusted to final weight with water. Due to the increasing chemical instability of ALA as pH is increased, the blend pH was not adjusted and, therefore, was around pH 2.

An amount (4.5 g) of aqueous blend was used to produce a film of area 15 cm² by slowly pouring the aqueous blend into a mould of internal dimensions 50 mm by 30 mm. The appropriate amount of ALA was dissolved directly into the aqueous blend immediately prior to casting. The mould, lined with release liner, siliconised side-up, attached with high vacuum grease, was placed on a leveled surface to allow the blend to spread evenly across the area of the mould. The cast blend was dried under a constant air flow at 25° C.

Films were removed from the mould by simply peeling the release liner, with attached film, off the base of the mould. The vacuum grease was then wiped off the non-siliconised side of the release liner. Bi-laminar bioadhesive patches were prepared by attaching, with the aid of gentle pressure, the exposed side of the films containing ALA, to equivalent areas of PVC backing films, prepared by heating Plastisol® emulsion to 160° C. for 15 minutes. For protection, the release liner was allowed to remain with its siliconised side attached to what had now become the release surface of the formed patch. Patches were then placed in moisture-impermeable poly(ester) foils, which were immediately heat sealed.

### Preparation of Bioadhesive Patches Containing ALA Using a Method According to One Embodiment of the Present Invention

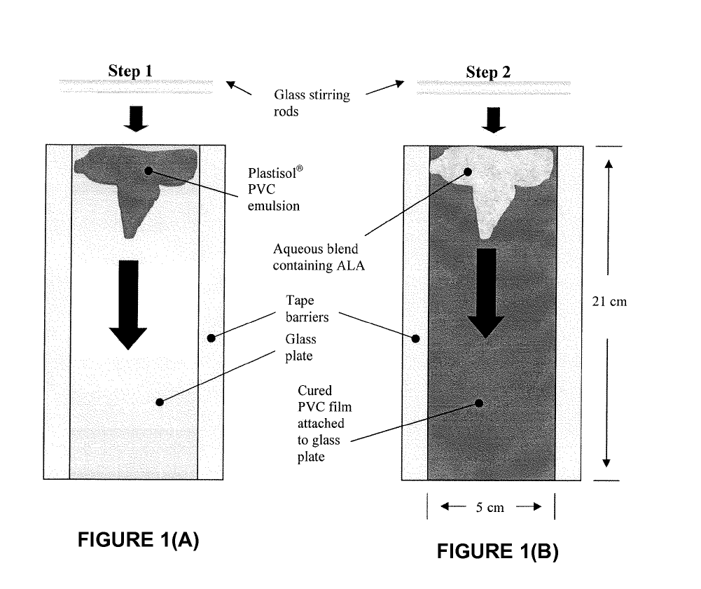

Two poly(vinyl chloride) films of rectangular dimensions 5 cm×21 cm were prepared as a first step. Plastisol® PVC emulsion (5 g) was placed on one end of a glass plate. Parallel runners, 200 μm in height and 21 cm long, were separated by a distance of 5 cm. Runners were prepared by attaching layers of Scotch™ tape, each 50 μm thick, adhesive side down, one on top of another, to build up a barrier of the required height. A glass stirring rod, with each end in continuous contact with a runner, was then used to smear the emulsion down the plate, as shown in FIG. 1 (A). In this way, PVC films, 200 μm thick, were produced. These films were then cured by heating at 160° C. for 15 minutes.

Five additional layers of tape were then added to each runner, such that the top of each barrier was 250 μm above the surface of the formed PVC films. An aqueous blend (3 g) of PMVE/MA and TPM, containing a defined loading of ALA and 30% w/w ethanol, was then placed at one end of each of the two PVC films. A glass stirring rod was then used to smear the semi-solids down the PVC films as shown in FIG. 1(B).

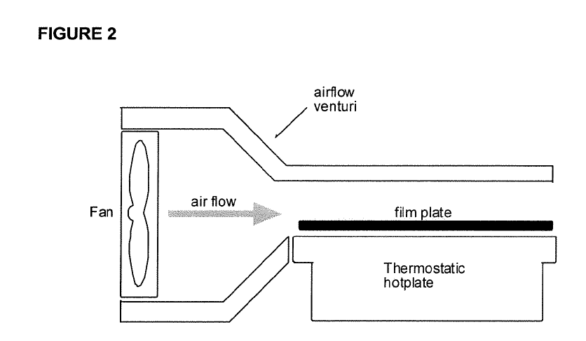

Thin films, produced in this way were then dried under a warm air flow for fifteen minutes in the specially designed film dryer shown in FIG. 2. An airflow venturi was constructed from Perspex and had three fans embedded into one end. The fans were used to draw in warm air from a blow heater and blow it over the drying film. The film was placed on a thermostatically controlled hot plate, normally used to dry electrophoresis gels. In this study the hot plate was not turned on since the blow heater gave a plate temperature of approximately 40° C. on its own.

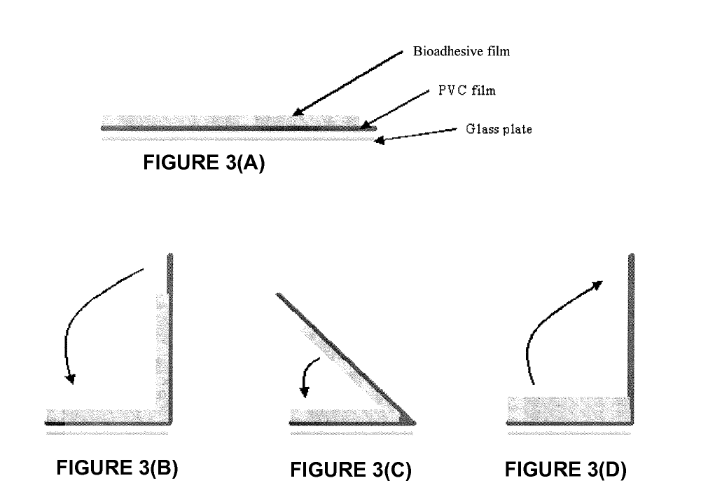

Each of the two, thin, bioadhesive films were then divided into sections having dimensions of 5 cm×3 cm. Each section was folded directly onto the one adjacent to it, gentle pressure applied and the PVC backing attached to the top section film peeled away so that the upper film was now bonded to the lower film. In this way, the lower film had its thickness doubled, as shown in FIG. 3.

This process was repeated until all sections had been folded on top of one another and bonded to produce one film, of dimensions 3 cm×5 cm, on each glass plate. The two films were then bonded to each other by the application of gentle pressure. A bi-laminar bioadhesive patch was then prepared by attaching one side of the film containing ALA, to an equivalent area of PVC backing, with the aid of gentle pressure. For protection, the siliconised side of an equivalent area of release liner was attached to the other surface of the formed patch. The patch was then placed in a moisture-impermeable poly(ester) foil which was immediately heat sealed.

### Drug-Distribution Studies of Loaded Patches

The ALA-loading in formed bioadhesive patches was determined by dissolving 1.5 cm² segments of patches in 10 ml 0.1 M borate buffer pH 5 (Pharmacopoeia Helvetica). The resulting dilute solution (1 ml) was then further diluted to 10 ml. This final solution was then analysed by HPLC, employing pre-column derivatisation with acetyl acetone and formaldehyde and fluorescence detection, as described previously (Donnelly et al., 2006). Results were expressed as mean ALA loadings per square centimetre of patch (±S.D.). Ten replicate measurements were initially made for each ALA loading to determine the homogeneity of ALA distribution in formed patches. In addition, patches subsequently prepared, for drug release studies, were selected at regular intervals and three 1.5 cm² segments assayed for ALA content. In this way, any variation in ALA loading between patches could be identified.

### Differential Scanning Calorimetry

Thermal analysis was carried out using a DSC Q100 Differential Scanning calorimeter (TA Instruments, Surrey, UK), calibrated for temperature and enthalpy using an indium standard. To determine an accurate melting point for ALA, 3.8 mg of the drug was placed in a hermetically sealed aluminium pan with an empty pan used as a reference. Subsequent analysis of bioadhesive films was performed at a heating rate of 2° C. minute⁻¹ over a temperature range to include the melting temperature of ALA (20-200° C.). In all cases, thermal analysis was performed no more than 48 hours after preparation. Results were reported as the mean (±S.D.) of five replicates.

### Bioadhesion Measurements

The bioadhesive properties of films, prepared from aqueous blends containing PMVE/MA and TPM loaded with ALA, were determined with respect to neonate porcine skin using a TA-XT2 Texture Analyser (Stable Microsystems, Haslemere, UK). Full thickness, shaved, neonate porcine skin was attached with cyanoacrylate adhesive to a lower platform. Film segments (1 cm²) were attached to the probe of the Texture Analyser using double-sided adhesive tape. Adhesion was initiated by adding a defined amount of water (10 μl) over an exposed skin sample (1 cm²) and immediately lowering the probe with attached film. Upon contact, a force of 5 N for 30 secs was applied before the probe was moved upwards at a speed of 0.1 mm s⁻¹. Adhesion was recorded as the force required to detach the sample from the surface of the excised skin. The distance to separation of a test film from the skin substrate, that is, the normal displacement from the skin surface that the probe had traveled at the instant the film and substrate lost contact with each other, was also recorded to provide some measure of the cohesion within the film sample. Results were reported as the mean (±S.D.) of five replicates.

### Determination of Tensile Properties

The tensile strength and percentage elongation at break of films prepared from aqueous blends containing PMVE/MA and TPM, and loaded with ALA, were determined using the Texture Analyser. Film strips of 5 mm width were grasped using an upper and lower flat-faced metal grip laminated with a smooth rubber grip. The distance between the grips was set at 20 mm and this distance, therefore, represented the length of film under stress. A cross-head speed of 6 mm s⁻¹ was used for all measurements.

The resultant force-time profiles were analysed using propriety software (Dimension 3.7E). Only results from films that were observed to break in the middle region of the test strip during testing were used. The percentage elongation at break, E_{b}, of tested films was determined using Equation 1 (Radebaugh, 1992).

$${E}_{b} = \frac{E}{{L}_{0}} 100 \qquad ( 1 )$$

Where E is the extension to break of the film and L₀ is its original length. The break strength, B, of tested films was determined using Equation 2 (Radebaugh, 1992).

$$B = \frac{F}{{A}_{R}} \qquad ( 2 )$$

Where F is the break force of the film and A_{R} is its cross-sectional area. Results were reported as the mean (±S.D.) of five replicates.

### Swelling Studies

The swelling and dissolution behaviour of ALA-loaded bioadhesive films was investigated, as described previously (McCarron et al., 2005). Segments of bioadhesive films, containing ALA, of area 4 cm², still attached to an equal area of release liner, were weighed using an electronic balance and individually placed in 50 ml of a 0.9% w/w saline solution. Segments were removed from the solution every 2.5 minutes, shaken to remove excess fluid and reweighed. Each experiment was performed for 45 minutes. At this time, any residual film on the release liner was removed, the liner dried by blotting with filter paper and weighed. This allowed calculation of the initial film weight. Results were reported as the mean (±S.D.) of five replicates.

### Drug Release Studies

The release of ALA from patch formulations was investigated using methods and the modified Franz cell apparatus described previously (McCarron et al., 2006). The orifice diameter in both donor and receptor compartments was 15 mm. Receptor compartment volumes, approximately 10 ml, were exactly determined by triplicate measurements of the weights of water they could accommodate. Account was taken for the volumes occupied by magnetic stirring bars. Compartment temperatures were kept constant at 37° C. by recirculating water from a thermostatically controlled bath. The receptor phase was 0.1 M borate buffer pH 5 (Pharmacopoeia Helvetica). This buffer was used since it was shown to maintain ALA stability at a high concentration (8 mg ml⁻¹) at temperatures up to 37° C. for periods of up to 6 hours (Donnelly, 2003). The buffer was degassed prior to use by vacuum filtration through a HPLC filter. Continuous stirring was provided by Teflon-coated stirring bars, rotating at 600 rpm. Stainless steel filter support grids were used to support Cuprophan® membranes. The membranes and support grids were sandwiched between the donor and receptor compartments. High vacuum grease and spring clips were used to hold the entire assembly together. The donor compartments were covered with laboratory film (Parafilm®).

Release from ALA-loaded patches was investigated by first cutting circular discs from 3 cm×5 cm patches using a sharp circular cork borer of inside diameter 1.5 cm. The bioadhesive surfaces of these discs were attached to the Cuprophan® membranes in the donor compartments using 10 μl of deionised water. Using a long needle, samples (0.25 ml) were removed from the receptor compartment at defined time intervals (5, 10, 15, 30, 60, 120, 180, 240, 300, 360 minutes). This volume was immediately replaced using blank, pre-warmed buffer. Samples removed were diluted to 5 ml with buffer and analysed by HPLC based on Oishi et al., (1996). Briefly, 50 μl of ALA sample was derivatised with an acetyl acetone and formaldehyde mixture. Solutions containing ALA derivative were injected onto a HPLC column (Spherisorb, 250 mm×4.6 mm, C18 ODS2 with 5 μm packing and fitted with a Spherisorb® S5 guard column; 10 mm×4.6 mm, C18 ODS2 with 5 μm packing, Waters associates, Harrow, UK). The mobile phase was 49.5% methanol/49.5% water/1% glacial acetic acid v/v/v, and the flow rate 1.5 ml min⁻¹. Detection was by fluorescence with excitation at 370 nm and emission at 460 nm (Shimadzu RF-535 fluorescence detector, Dyson Instruments Ltd, Tyne & Wear, UK). The chromatograms obtained were analysed using proprietary Shimadzu Class VP™ software. Results were reported as the means (±S.D.) of three replicates.

### Results

In preparing ALA-loaded bioadhesive films by conventional casting into glass moulds, the ALA powder was simply dissolved with stirring in the aqueous blend immediately before casting. To produce a film containing 38 mg ALA cm⁻², for example, 0.57 g of ALA was dissolved in the 4.5 g of aqueous blend that would be used to produce a drug free film of dimensions 3 cm×5 cm. The entire formulation was then cast into the glass mould and dried under a constant warm air flow. Films containing 19 and 50 mg cm⁻² ALA, respectively, were produced by dissolving 0.285 g and 0.75 g, respectively, in 4.5 g of aqueous blend.

The casting method was associated with a number of problems. Stirring the ALA into the casting blend introduced air bubbles and these were difficult to remove from the aiming film, due to its increasing viscosity. In addition, formulations typically took at least 48 hours to dry completely. At this stage, some of the ALA in the bioadhesive films containing 38 and 50 mg cm⁻² ALA had come out of solution, leaving the film white in colour and with a textured surface.

Initial attempts at reducing incorporation of air bubbles, reducing drying time, and preventing ALA crystallisation met with little success. Pouring concentrated aqueous solutions of ALA onto pre-formed drug-free films cast from blends containing 20% w/w PMVE/MA and 10% w/w TPM led, upon drying, to deposition of ALA crystals on the surfaces of the films, which were now rough and non-adhesive. Another approach involved dividing the ALA-containing casting blend into five portions of equal weights. Each portion was cast into the mould, one on top of the other, once the bottom portion had dried to produce a thin film. This method was unsuccessful, in that each successive layer cast redissolved the layer cast before it. The end result was a film that still took 48 hours to dry. Changing the composition of the casting blend, so that it now contained 30% w/w ethanol, was aimed at producing a film that dried more quickly. This reduced the water content in the blend to 40% w/w. A “custard skin” effect was observed, with the surface of the blend drying quickly and then retarding the drying of the fluid beneath. Freeze-drying of blends led to similar results, except that the “skin” overlying the fluid expanded to produce a balloon-like structure.

Films prepared by the novel multiple lamination method according to the invention were dry to the touch in 15 minutes. Folding to produce the final patch took approximately 10 minutes. No air bubbles or solid drug were evident in the formed films. Over-drying of such films, by drying for 25-30 minutes, caused ALA to come out of solution in the film matrix.

Films dried for 15 minutes and folded to produce patches were clear and showed no evidence of ALA coming out of solution. After several (>7) days of storage (5° C. to improve ALA stability), some ALA was observed to come out of solution in patches containing 38 and 50 mg cm⁻² ALA. Patches containing 19 mg cm⁻² ALA, however, were still transparent and showed no evidence of solid ALA, even after 12 months of storage.

The multiple lamination method employed a long, shallow mould, to produce long, thin films quickly. Since the dimensions of this mould were 250 μm high times 5 cm wide times 21 cm long, the volume was 2.625 ml. To prepare a film of dimensions 3 cm×5 cm, containing 38 mg ALA cm⁻², 0.57 g of ALA would be needed. Assuming 1 g of ALA occupies 1 ml in solution and knowing that each patch was prepared in two halves, 0.285 g would be added to 2.34 g of gel to produce an aqueous blend for each half of the patch. To allow for spreading of the blend during the smearing process, slight excesses were used such that 0.33 g of ALA and 2.67 g of aqueous blend were used for each half of the patch. The weights of ALA and aqueous blends required to produce patches with ALA-loadings of 19 and 50 mg cm⁻², respectively were calculated in a similar way.

The mean loadings of ALA in patches prepared by both the multiple lamination and the casting methods are shown in Table 1. The ALA loadings in the patches prepared by the two different methods were not significantly different from each other (p<0.0001). Patches subsequently assayed did not show significant differences (p<0.0001) in their ALA-loadings from the mean values shown in Table 1.

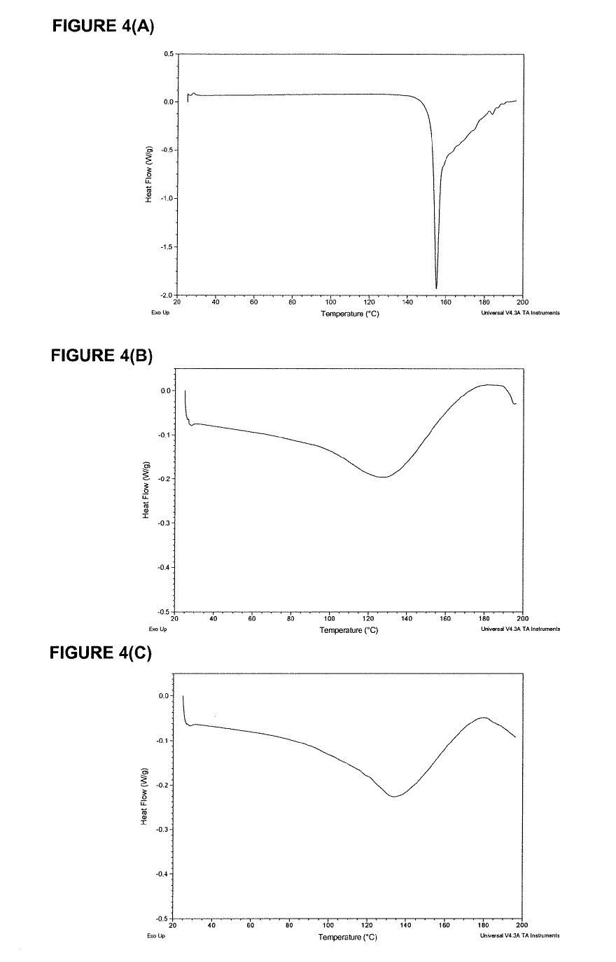

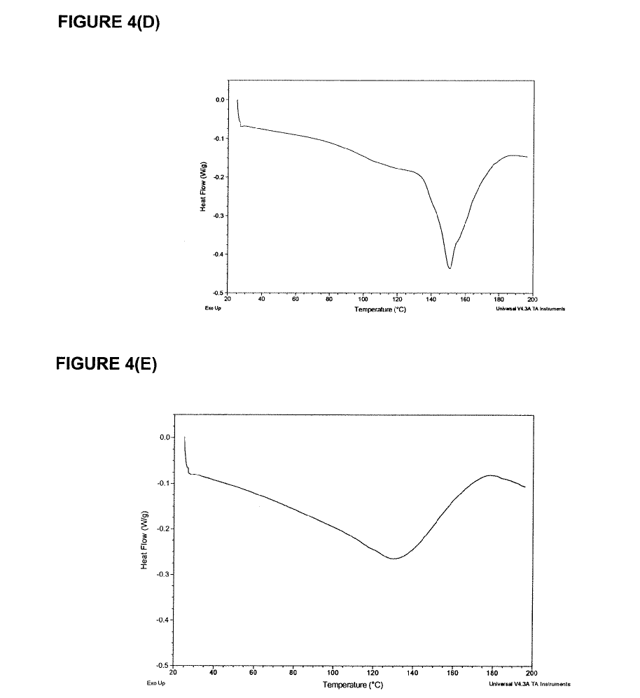

FIG. 4 A shows a typical DSC trace for ALA, where the endotherm corresponding to the ALA melt is observed at approximately 155° C. Thermal analyses of cast and folded films void of ALA revealed that no significant background events are present around 155° C. (FIGS. 4 B and 4 C, respectively). Films containing ALA prepared by the casting method showed clearly-defined melts at loadings of 38 mg cm⁻² and 50 mg cm⁻² (FIG. 4 D). However, no endotherm corresponding to the ALA melt was observed for folded films at any of the concentrations prepared (FIG. 4 E).

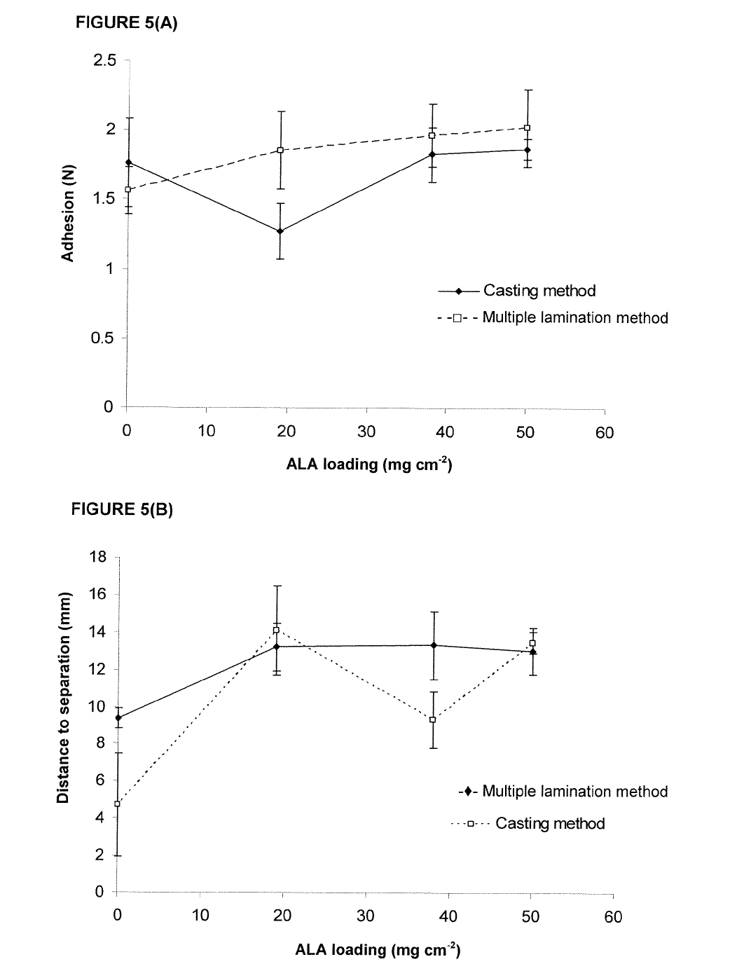

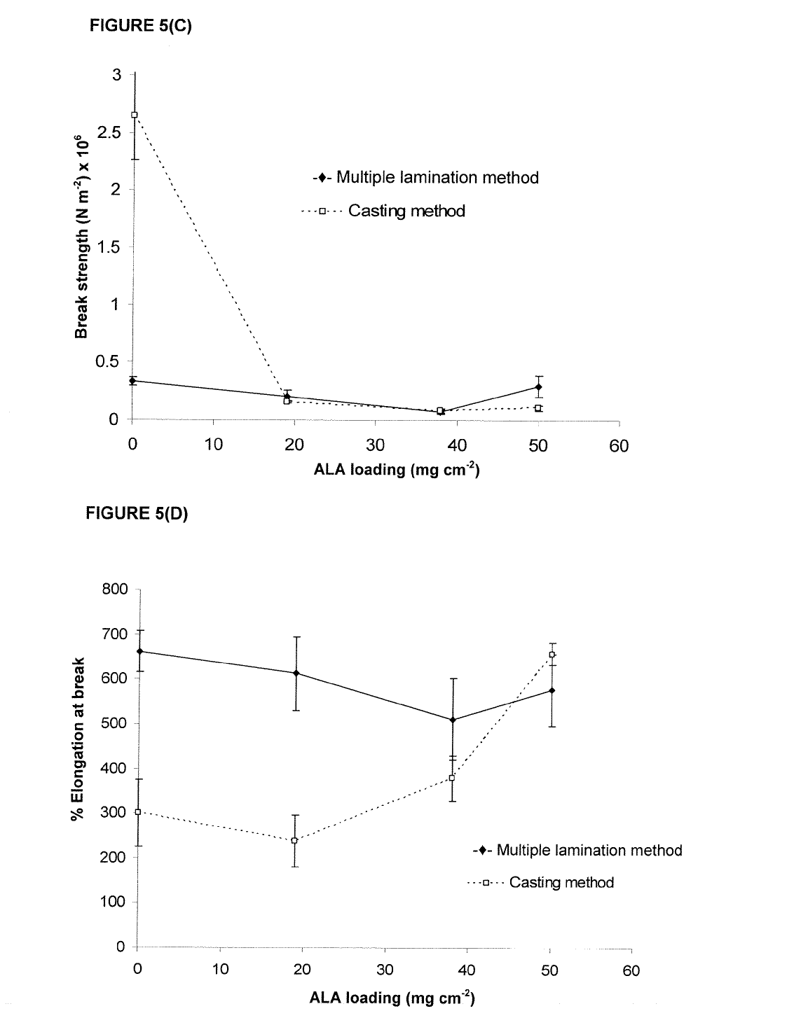

Bioadhesion, to shaved neonate porcine skin, was not significantly affected by method of patch preparation (p=0.0735) or ALA loading (p=0.7778 for the multiple lamination method, p=0.4356 for the casting method), as can be seen from FIG. 5 A. FIG. 5 B shows the influence of ALA loading and method of preparation on the distance to separation of 1 cm⁻² film segments under test and shaved neonate porcine skin.

The mean distance to separation of films cast from blends containing 20% w/w PMVE/MA and 10% w/w TPM increased significantly with the addition of ALA (p<0.0001). Further increasing the ALA loading from 19 to 38 mg cm⁻² (p=0.0017) and from 38 to 50 mg cm⁻² (p=0.0050), respectively, did not cause any further significant increases in distance to separation. The mean distance to separation of drug-free films prepared by the multiple lamination method was significantly greater than that of corresponding films prepared by casting (p=0.0307). Again, a significant increase in distance to separation was observed with the inclusion of ALA (p=0.0021). In addition, increasing the ALA loading from 19 to 38 mg cm⁻² (p=0.7462) and from 38 to 50 mg cm⁻² (p=0.91), respectively, did not cause any further significant increases in distance to separation. There were no significant differences in mean distances to separation observed between ALA-loaded films prepared by either of the two methods (p=0.3355).

Table 2 shows the influences of ALA-loading and method of preparation on the mean thicknesses of bioadhesive films. The mean thickness of films cast from blends containing 20% w/w PMVE/MA and 10% w/w TPM increased significantly with the addition of ALA (p<0.0001). Further increasing the ALA loading from 19 to 38 (p=0.27) and from 38 to 50 mg cm⁻² (p=0.231) did not cause significant increases in film thickness. The mean thickness of drug-free films prepared by the multiple lamination method was significantly greater than that of corresponding films prepared by casting (p=0.0065). However, no significant increase in film thickness was observed with the inclusion of ALA (p=0.4822). In addition, increasing the ALA loading from 19 to 38 mg cm⁻² (p=0.27) and from 38 to 50 mg cm⁻² (p=0.34), respectively, did not cause any significant increases in film thickness. There were no significant differences in mean thicknesses observed between ALA-loaded films containing 19 (p=0.4822) or 38 mg ALA cm⁻² (p=0.0683) prepared by either of the two methods. However, cast films containing 50 mg cm⁻² were significantly thicker than the corresponding folded films (p=0.0183).

As can be seen from FIG. 5 C, the addition of ALA caused a significant decrease in break strength of cast films (p<0.0001). Further increasing the ALA loading from 19 mg cm⁻² to 38 mg cm⁻² (p=0.2882) and from 38 mg cm⁻² to 50 mg cm⁻² (p=0.6850), respectively, did not cause any further significant reductions in break strengths. Drug-free films prepared by the multiple lamination method had significantly lower break strengths than the corresponding cast films (p<0.0001). ALA addition reduced the break strengths of folded films still further (p=0.0026). However, increasing the ALA loading from 19 mg cm⁻² to 38 mg cm⁻² (p=0.28) and from 38 mg cm⁻² to 50 mg cm⁻² (p=0.0519), respectively, did not cause any further significant reductions in break strengths. Moreover, the break strengths of ALA-loaded folded films were not significantly different from the corresponding films prepared by casting (p=0.36).

As can be seen from FIG. 5 D, increasing the ALA content of cast films from 0 to 19 mg cm⁻² had no significant influence on their percentage elongations at break (p=0.0638). Increasing the ALA loading from 19 mg cm⁻² to 38 mg cm⁻² (p=0.0008) and from 38 mg cm⁻² to 50 mg cm⁻² (p<0.0001), respectively, caused significant increases in percentage elongations at break. Drug-free films, prepared by the multiple lamination method, showed significantly greater percentage elongations at break than the corresponding cast films (p<0.0001). ALA addition caused no further significant increases in percentage elongations at break of folded films. The percentage elongations at break of folded and cast films containing 50 mg ALA cm⁻² were not significantly different (p=0.22).

Table 3 shows the influence of ALA loading and method of preparation on the swelling and dissolution behaviour of bioadhesive films. As can be seen from Table 3, increasing ALA loadings had no significant effect on the maximum swollen weights of films prepared by casting or multiple lamination methods. ALA-loaded films, however, achieved their maximum swollen weights in 2.5 minutes. Drug-free films did not achieve their maximum swollen weights until 5 minutes after immersion.

From Table 3 it may be seen that, as the ALA loading in cast films was increased from 0 to 19 mg cm⁻² (p=0.0495), from 19 to 38 mg cm⁻² (p=0.0462) and from 38 to 50 mg cm⁻² (p<0.0001), respectively, significant reductions were observed in the weights of films after 45 minutes immersion. A similar pattern was observed for films prepared by the multiple lamination method in that as the ALA loading was increased from 0 to 19 (p<0.0001), from 19 to 38 mg cm⁻² (p=0.0102) and from 38 to 50 mg cm⁻² (p=0.0182), respectively, significant reductions were observed in the weights of films after 45 minutes immersion. The maximum swollen weights of ALA-loaded and drug free films prepared by multiple lamination and casting methods were not significantly different from each other. However, the weights of ALA-loaded films containing 19 (p=0.0031), 38 (p<0.0001) and 50 mg cm⁻² (p=0.0488), respectively, prepared by the multiple lamination method were significantly less than those of the corresponding films prepared by casting after 45 minutes immersion. There was no significant difference between final weights of the drug free films prepared by the two methods.

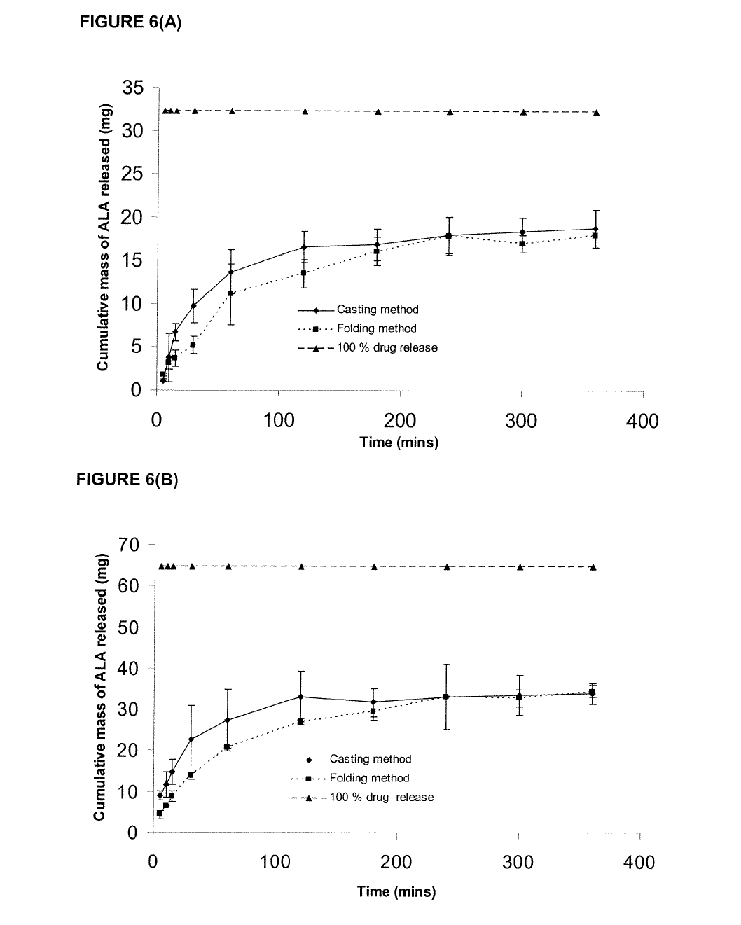

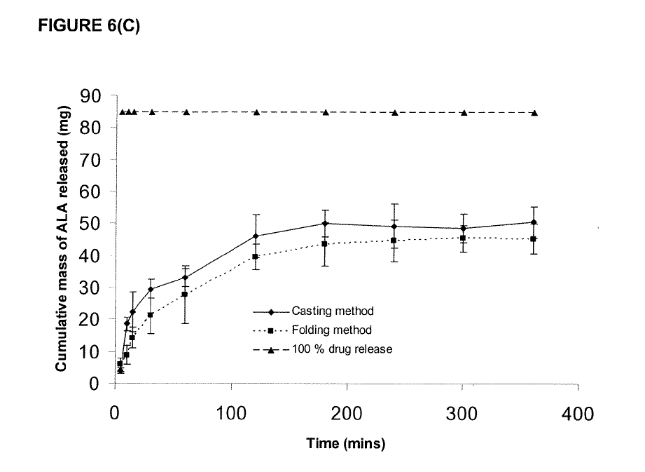

The release profiles of patches based on films produced both by the casting and multiple lamination methods are shown in FIGS. 6 A-C. From Table 4, is can be seen that as the drug loading was increased from 19 to 38 mg cm⁻² (p<0.0001 for cast patches, p<0.0001 for multiple laminate patches) and from 38 to 50 mg cm⁻² (p<0.0001 for cast patches, p<0.0001 for multiple laminate patches), respectively; the amount of ALA released after 6 hours increased significantly for both methods of production. The method of film production was found to have no significant influence on drug release, regardless of drug loading. All patches had released 52-59% of their drug loadings across Cuprophan® membranes over 6 hours (Table 4).

A number of methods for production of bioadhesive patches containing 5-aminolevulinic acid (or salt thereof) (ALA) were investigated in the present study. However, only the multiple lamination method produced films that were deemed suitable for inclusion in a bi-laminar patch (i.e. a layered patch containing two layers) for topical ALA delivery. Patches based on films prepared by all other methods were associated with significant problems. Only the conventional casting method produced ALA-containing films that were even suitable for comparison with folded films. However, these cast films often contained air bubbles, which were difficult to eliminate. Films prepared by the multiple lamination method were dry in 15 minutes and only took 10 minutes to fold into the final patch backed with a PVC film. Films were clear, even when loaded with 50 mg cm⁻² ALA and no air bubbles were visible. If dried excessively, or if left standing for several days, the films containing 38 and 50 mg cm⁻² ALA, did show evidence of solid drug deposition. Films containing 19 mg cm⁻² ALA did not show any evidence of solid drug even on storage for 12 months.

The determined ALA loadings in films containing theoretical ALA loadings of 19, 38 and 50 mg cm⁻², prepared by casting or multiple lamination, were not significantly different from each other. In all cases, standard deviations were less than 10% of the mean loading, indicating a homogenous distribution of ALA in the films. Films prepared subsequently did not differ significantly in their ALA loadings from those initially prepared.

Thermal analysis revealed that the melting point for ALA is 155° C., which corresponds closely to literature vales (Merck Index 14^{th} Edition). Thermograms for films void of ALA produced by either the multiple lamination or casting methods displayed broad endotherms over the range of 100-50° C., relating to moisture loss from the sample (Ford, (1999). However, as expected, blank films lacked the well-defined endothermic peak at 155° C. associated with the ALA melt. Cast films containing 38 and 50 mg cm⁻² appeared white to the naked eye, indicating that some ALA had crashed out of solution. This observation was confirmed by DSC, whereby the endotherm associated with the ALA melt was clearly distinguishable. At the lowest drug loading of 19 mg cm⁻², no endotherm was observed, indicating that the drug is maintained in solution. In contrast, films produced by the multiple lamination method were clear at all three concentrations of ALA, and no melting endotherm was observed for ALA.

The force required to remove ALA-containing films from pre-wetted neonate porcine skin was not significantly affected by ALA loading or method of preparation. The mean distance to separation of both cast and folded films significantly increased with the addition of ALA. Further increasing their ALA contents did not cause any further increases in their mean distances to separation. Once the ALA content was increased above 19 mg cm⁻² it may have exceeded its maximum plasticising capabilities. The increased distance to separation of drug-free folded films compared to the corresponding cast films may be due to the folded films containing more water as water is capable of plasticising polymers. Hence, these films had reduced internal cohesion and, consequently increased distances to separation.

Drug-free films prepared by the multiple lamination method were significantly thicker than those prepared by casting. This may be as a result of the laminating process causing air to become entrapped between layers or, because the folded films contain more water. ALA-containing cast films were significantly thicker than the corresponding drug-free films. This may be due to the high ALA loadings or to the hygroscopic nature of ALA causing more water to be retained by the film. Film thicknesses for ALA-containing films prepared by the two methods were not significantly different, regardless of drug loading. This may be because the high ALA loadings, combined with the possible water-retaining effect of ALA, have a greater influence on final film thickness than method of preparation.

Drug-free films prepared by the multiple lamination method showed significantly lower weights after 45 minutes immersion in 0.9% w/w saline than the corresponding cast films. This may be due to the greater contribution of water to the initial weights of the folded films. As ALA-loading was increased, in both folded and cast films, their final weights after 45 minutes immersion showed corresponding significant decreases. This may be due to the increasingly significant contributions made to their initial weights by the highly water soluble ALA, which may rapidly dissolve out of the films. Alternatively, the hygroscopic ALA may draw water into the films and, hence enhance dissolution. Increasing the content of tripropylene glycol methyl ether, increased the dissolution of films cast from aqueous blends containing PMVE/MA. That the weight loss of ALA-containing films after 45 minutes immersion was independent of preparation method was likely to be due to the fact that the ALA loadings were so high that any contribution made by the method of preparation to dissolution was offset. The increased dissolution of ALA-loaded films, with respect to the corresponding drug-free films, may be of concern. This may affect their in vivo performance, in that on drawing moisture from the body, they may become gel-like and become difficult to keep in place for the desired time period.

The influence of film preparation method on ALA release was assessed in vitro using the Franz Cell Model, employing Cuprophan® as a model membrane. ALA remains in solution in films produced by the casting method at a concentration of 19 mg cm⁻². However, when the concentration is doubled, some ALA is found to crystallise out. This indicates that the saturation solubility of ALA in these films lies between 19 and 38 mg cm⁻². ALA remains in solution in folded films at concentrations above the saturated solubility of ALA. Therefore these formulations may be said to be supersaturated. In supersaturated systems, the thermodynamic activity of the drug in the vehicle is increased above unity, thus enhancing the drive for drug delivery However, no significant difference was observed in the drug release profiles from films prepared by the two methods. Cuprophan® is a dialysis type membrane with a molecular cut-off of approximately 10,000 daltons. Although Cuprophan® acts as a semi-permeable membrane to ALA diffusion; it also allows water ingress into the donor compartment of the Franz Cell. In the case of cast films, such water uptake rapidly dissolves the highly water soluble ALA, which is then in solution and available for diffusion. Similarly, water uptake will have a significant influence on the release from folded films. When the supersaturated folded films take up water, their volume will be increased and the concentration of ALA in solution reduced. As a result, the concentration drive for diffusion will be reduced, reverting to a situation similar to the swollen cast films. In vivo, a similar situation would be expected, as the occlusive nature of the PVC backing layer is likely to induce significant sweating of the underlying skin. When patches are applied to a naturally moist area, such as the oral cavity or vaginal epithelium, the fluids present will have a similar effect. The hydrophilic matrix of the patch will lead to fluid ingress and a significant dilution effect, thus negating the penetration enhancing characteristics of the originally supersaturated folded system. However, for a less water soluble drug than ALA, this may not be the case and supersaturation may be maintained during application, leading to enhanced drug delivery.

### EXAMPLE 2

### Volatile Drug

### Materials and Methods

### Preparation of Bioadhesive Patches Containing Nicotine by Casting

In order to correspond closely to commercially available nicotine transdermal patches, a theoretical drug loading of 10.4 mg cm⁻² was chosen. Patches were prepared by the casting method, as described in 2.2 above, with appropriate amounts of nicotine replacing ALA in the casting blends.

### Preparation of Bioadhesive Patches Containing Nicotine by a Method According to an Embodiment of the Present Invention

Patches were initially prepared using the multiple lamination method according to an embodiment of the invention from aqueous blends containing 30% w/w ethanol, as described in 2.3 above for ALA. Patches were also prepared from aqueous blends containing 22% w/w acetone. These organic solvent concentrations were the highest concentrations which still allowed films to form properly. Finally, the thickness of the barrier used to prepare films for folding into patches was also varied, with aqueous blends now containing neither ethanol nor acetone.

### Determination of Nicotine Loadings in Formed Patches

Defined areas (1.0 cm²) were removed from formed patches and dissolved in 10.0 ml deionised water. Samples were then diluted appropriately and filtered through 0.45 μm and 0.22 μm syringe filters before determination by UV spectroscopy at 260 nm. Nicotine loadings in each type of patch were reported as the mean (±S.D.) of five replicates.

### Statistical Analysis

Where appropriate, data was analysed using a one-way, Analysis of Variance (ANOVA). Post-hoc comparisons were made using Fisher's PLSD test. In all cases, p<0.05 denoted significance.

### Results

Films containing nicotine produced using the casting method took approximately 48 hours to dry. All nicotine-containing films prepared using the multiple lamination method, whether containing an organic solvent or not, were dry in less than 15 minutes. For films prepared when the barrier height was 50 μm or 150 μm it was, obviously, necessary to increase the lengths of the films appropriately. Theoretically, this meant that, when folded, the final patch contained 10.4 mg nicotine cm⁻² in each case.

As can be seen from Table 5, films prepared using the casting method had lost approximately 50% of their theoretical nicotine loading upon completion of drying at 48 hours. Films prepared by the multiple lamination method from aqueous blends containing ethanol or acetone, while dry in less than 15 minutes, had also lost significant proportions of their theoretical nicotine loading upon completion of folding into patches. Patches prepared by the multiple lamination method from aqueous blends containing neither acetone nor ethanol lost only approximately 10% of their theoretical nicotine loadings upon drying. Patches prepared using a barrier height of 250 μm retained the highest proportion of nicotine (93.45%). The nicotine loading of patches prepared using a barrier height of 50 μm showed the greatest variability. Preparation of these patches was problematic, due to the very thin nature of the films formed (<50 μm), which made handling difficult. In addition, very long films (105 cm) were required to produce a patch that, upon folding, contained an equivalent drug loading to that prepared using a barrier height of 250 μm. This made these films very difficult to manipulate and mistakes were frequent.

As expected, drying cast films over 48 hours led to extensive loss of nicotine. Commercially available transdermal nicotine patches are based on pressure sensitive adhesive matrices cast from organic solvents. Such systems typically are dry in less than 5 minutes, meaning extensive loss of this relatively volatile drug does not occur. Addition of ethanol and acetone to aqueous blends was unsuccessful in preventing significant nicotine loss from films, which still took 15 minutes to dry. It is likely that the organic solvents reduced the boiling points of the aqueous blends, encouraging evaporation and nicotine loss. Due to its greater volatility, acetone (bp 56.5° C.) had a more pronounced effect on nicotine loss than ethanol (bp 78.5° C.) (Merck Index). However, as both blends contained a high proportion of water (bp 100° C.), the overall drying time of the films was not significantly reduced, with the organic solvents likely to have completely evaporated before sufficient water was lost to produce a dry film. Reducing barrier height did not significantly reduce drying time. However, reducing film thickness necessitated significant increases in film length. This made the process significantly more time consuming. Films prepared from aqueous blends containing neither acetone nor ethanol with a barrier height of 250 μm were dry in 15 minutes and had been folded into completely formed patches within a further 10 minutes. Moreover, the majority of the incorporated nicotine remained within the patch. The absence of volatile organic solvents meant that evaporation of nicotine was not enhanced.

Thus by way of the present invention it has been shown for the first time that a multiple lamination procedure can produce bioadhesive patches in a fraction of the time required using the conventional casting approach. Patches containing a drug at high loading (ALA) were dry in 15 minutes with no evidence of crystallization, due to the production of a saturated solution during rapid drying. Patches containing a volatile drug (nicotine) were also dry in 15 minutes and >90% of their drug loading was retained. This procedure could readily be adapted for automation by industry.

### EXAMPLE 3

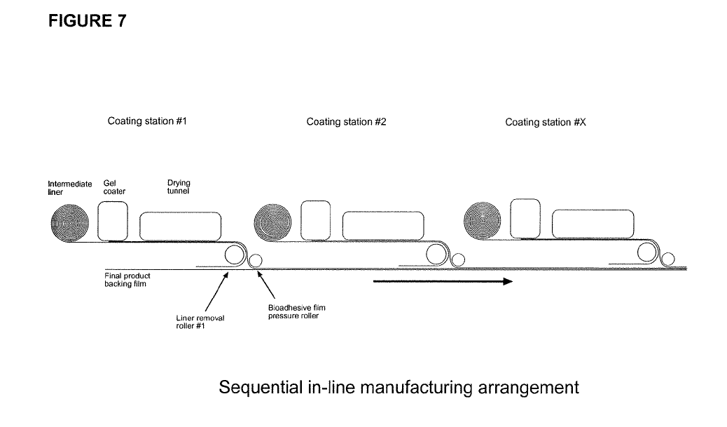

The patch may be assembled by way of a sequential in-line manufacturing arrangement as set out in FIG. 7 in which the patch is assembled using a sequence of coating and drying stations. Coating stations may be conventional film-applying coating stations, where a film according to the present invention may be produced.

The number of coating and drying stations present in the manufacturing arrangement is dependent on the number of layers to be included in the patch. For example, if the patch is made up of seven thin bioadhesive layers, then seven stations are required.

Each station is fed with an intermediate backing layer that runs under a coating device that applies a thin layer of drug-containing polymeric solution (such as Gantrez solution) thereon. The polymeric solution may also be presented in the form of a gel for subsequent coating onto the backing layer. Suitable coating devices include a knife coater or other similar device(s) known in the coating industry.

The bilayer formed runs under a drying device, such as a heated air tunnel, which reduces the polymeric layer to a non-flowable tacky film. As that layer is applied thinly, drying in such a way is feasible and indeed particularly advantageous. The tacky film is then separated from the intermediate backing layer and applied to a final product backing layer using a form of pressure roller. This produces a new bilayer that proceeds to the next coating station.

Coating station 2 operates in the same way as 1. This time, the tacky drug-containing layer is separated from its intermediate backing layer and applied, again by pressure roller to the new bilayer passing underneath. This produces a trilayer—two adhesive drug-containing layers and a final product backing layer.

This process is repeated as required, with each pass through a coating station applying a new thin polymeric layer. It should be noted that such a method does not involve the application of wet layers applied on top of one another, but instead, a series of semi-solid tacky layers are adhered together under mild pressure.

### EXAMPLE 4

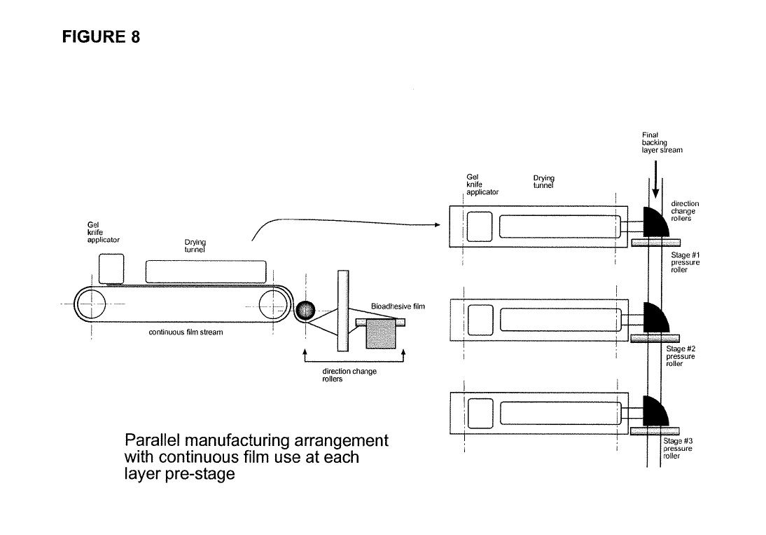

An alternative patch manufacturing arrangement is set out in FIG. 8 in which the patch is assembled using a parallel manufacturing arrangement with continuous film use at each layer pre-stage.

In such an arrangement the coating stations are positioned sequentially rather than in a parallel arrangement and will, therefore, not take up so much room. Furthermore in such an arrangement the intermediate backing layer in each station is recycled around two rollers. The coating device applies a thin layer of drug-containing polymeric solution or gel, which is then dried to the required tackiness. A series of rollers then remove this bilayer and changes its direction so that it can be applied to a final product backing layer. This bilayer runs at 90 degrees to each station.

Each station produces a bilayer that is applied to a final product backing layer, with pressure rollers ensuring firm contact and removal of all traces of air. Such a method means minimal wastage of intermediate backing substrate and will also conserve space.

It is envisaged that other manufacturing arrangements could also be used such as for example but not limited to one wherein all the coating stations are amalgamated into a large station which could run seven or so parallel tracks simultaneously. Whatever the arrangement in order to fulfil the requirements of the invention it must incorporate the step(s) of at least forming a thin tacky drug-containing polymeric layer and pressing several of these together, one on top of the other, to give a final bioadhesive layer or patch.

In any event due to the reduced time, energy and ensuing finance now required, the procedure developed could lead to bioadhesive patch-based drug delivery systems becoming commercially viable. This would, in turn, mean that pathological conditions occurring in wet or moist areas of the body could now be routinely treated by prolonged site-specific drug delivery, as mediated by a commercially produced bioadhesive patch.

### Tables

TABLE 1
Influence of preparation method on the ALA-loading of bioadhesive
films prepared from aqueous, or aqueous alcoholic, blends containing
20% w/w PMVE/MA and 10% w/w TPM. Results are reported as the
means (±S.D.) of ten replicate samples taken from single films.

| Theoretical | | |
|---|---|---|
| ALA-loading | ALA-loading for cast films | ALA-loading for folded films |
| (mg cm⁻²) | (mg cm⁻²) (±S.D.) | (mg cm⁻²) (±S.D.) |
| 19 | 19.18 ± 0.96 | 20.10 ± 1.74 |
| 38 | 40.14 ± 1.56 | 39.55 ± 3.40 |
| 50 | 49.79 ± 4.33 | 51.84 ± 2.48 |

TABLE 2
Influences of ALA loading and method of preparation on thicknesses
of bioadhesive films prepared from aqueous, or aqueous alcoholic,
blends containing 20% PMVE/MA and 10% w/w TPM
(mean ± S.D., n = 5).

| Theoretical | | |
|---|---|---|
| ALA-loading | | Thickness (mm) of folded |
| (mg cm⁻²) | Thickness (mm) of cast films | films |
| 0 | 0.49 ± 0.02 | 0.81 ± 0.06 |
| 19 | 0.86 ± 0.07 | 0.78 ± 0.11 |
| 38 | 0.84 ± 0.11 | 0.79 ± 0.06 |
| 50 | 0.85 ± 0.10 | 0.80 ± 0.05 |

TABLE 3
Influence of ALA loading and method of preparation on the swelling and dissolution behaviour of bioadhesive films prepared
from aqueous, or aqueous alcoholic, blends containing 20% w/w PMVE/MA and 10% w/w TPM (mean ± S.D., n = 5).

| ALA | Maximum weight of | Time to Maximum | Weight of cast films | Maximum weight | Time to Maximum | Weight of folded |
|---|---|---|---|---|---|---|
| loading | cast films | weight of cast | after 45 minutes | of folded films | weight of folded | films after 45 minutes |
| (mg cm⁻²) | (% of original weight) | films (minutes) | (% of original weight) | (% of original weight) | films (minutes) | (% of original weight) |
| 0 | 121.16 ± 1.13 | 5.0 | 83.06 ± 6.36 | 125.75 ± 3.16 | 5.0 | 87.80 ± 4.62 |
| 19 | 119.85 ± 3.99 | 2.5 | 77.61 ± 3.79 | 134.38 ± 3.44 | 2.5 | 65.68 ± 6.31 |
| 38 | 115.93 ± 3.49 | 2.5 | 76.34 ± 2.4 | 121.04 ± 7.45 | 2.5 | 60.63 ± 3.96 |
| 50 | 116.24 ± 2.96 | 2.5 | 58.71 ± 6.19 | 139.90 ± 5.87 | 2.5 | 52.96 ± 6.03 |

TABLE 4
Percentages of total ALA loadings released from patches across
Cuprophan ® membrane after 6 hours. (means ± S.D., n = 5).

| | | | |
|---|---|---|---|
| | Theoretical ALA- | Cumulative Mass | Percentage of total |
| | loading in | ALA released | ALA released |
| | 1.77 cm² | after 6 | after 6 |
| Formulation | (mg) | hours | hours |

Casting

| | | | |
|---|---|---|---|
| 19 mg cm⁻² | 33.56 | 18.78 ± 2.17 | 58.16 ± 6.74 |
| 38 mg cm⁻² | 67.15 | 33.97 ± 2.54 | 52.60 ± 3.93 |
| 50 mg cm⁻² | 88.36 | 50.54 ± 5.03 | 59.54 ± 5.92 |

Multiple lamination

| | | | |
|---|---|---|---|
| 19 mg cm⁻² | 33.56 | 17.99 ± 1.34 | 55.72 ± 4.16 |
| 38 mg cm⁻² | 67.15 | 34.53 ± 1.39 | 53.45 ± 2.16 |
| 50 mg cm⁻² | 88.36 | 45.36 ± 4.7 | 53.36 ± 5.54 |

TABLE 5
Influence of preparation method on nicotine remaining in
bioadhesive patches (means ± S.D., n = 5).

| | Nicotine loading | Mean % of | |
|---|---|---|---|
| | determined | theoretical | Mean % drug |
| Method employed | (mg cm⁻²) | loading | lost |
| Casting | 5.02 ± 0.65 | 48.28 | 51.72 |
| Multiple lamination | | | |
| 30% w/w ethanol in | 6.42 ± 0.61 | 61.73 | 38.27 |
| aqueous blend | | | |
| 22% w/w acetone in | 3.64 ± 0.19 | 35.04 | 64.96 |
| aqueous blend | | | |
| Barrier 50 μm high | 9.40 ± 1.87 | 90.35 | 9.65 |
| Barrier 150 μm high | 9.32 ± 1.21 | 89.62 | 10.38 |
| Barrier 250 μ m high | 9.72 ± 0.68 | 93.45 | 6.55 |

1. A method of making a moist, layered bioadhesive patch, comprising:
(a) providing an aqueous solution comprising at least one polymer selected from the group consisting of poly(methyl vinyl ether/maleic acid) and esters/amides thereof, poly(methyl vinyl ether/maleic anhydride) (PMVE/MA) and esters/amides thereof, and poly(acrylic acids) and esters/amides thereof;
(b) spreading out or spraying a thin layer of the solution resulting from (a) to form a film;
(c) drying the film formed;
(d) applying a first layer of the film on a backing;
(e) applying a second layer of the film on the first layer;
(f) pressing the first layer and second layer together until the layers adhere.

2. A method of making a moist, layered bioadhesive patch according to claim 1, wherein the step of applying the second layer of the film on the first layer comprises folding the first layer onto itself to form a second layer on the first layer, the method further comprising removing at least a portion of the backing layer to expose part of the second layer after the second layer has been adhered to the first layer.

3. A method of making a moist, layered bioadhesive patch according to claim 1, further comprising repeating steps (e) and (f) to build up the patch to a desired thickness.

4. A method according to claim 3, wherein the patch contains 3-10 film layers.

5. A method according to claim 1, wherein the thickness of each film layer is from 1 μm to 500 μm.

6. A method according to claim 1, wherein the thickness of each film layer is from 25 μm to 75 μm.

7. A method according to claim 1, wherein the total thickness of the patch is from of 2 μm to 5000 μm.

8. A method according to claim 1, wherein each film layer exhibits a tensile strength greater than 1.0×10⁻⁸N cm⁻² and a residual tackiness, such that detachment of two layers of the same material requires a force of removal greater than 1.0 N cm⁻².

9. A method according to claim 1, wherein the aqueous solution of (a) further comprises a pharmaceutically active compound.

10. A method according to claim 9, wherein the pharmaceutically active compound is selected from the group consisting of nicotine, 5-ALA and derivatives thereof, antibiotics, parasympatholytics, cholinergics, neuroleptics, antidepressants, antihypertensives, photosensitisers, photosensitiser precursors, sympathomimetics, sympatholytics and antisympathotonics, antiolytics, local anaesthetics, central analgesics, antirheumatics, coronary therapeutics, hormones, antihistamines, prostaglandin derivatives, vitamins, nutrients and cytostatics.

11. A method according to claim 1, wherein the aqueous solution of (a) further comprises at least one plasticizer chosen from among glycerol, propylene glycol, poly(ethylene glycol) and tripropylene glycol monomethyl ether (TPM).

12. A method according to claim 11, wherein the plasticizer is tripropylene glycol monomethyl ether (TPM) and is present in an amount in the range of 0.25 to 25% w/w of the aqueous solution.

13. A method according to claim 1, wherein the aqueous solution further comprises a water-miscible co-solvent.

14. A method according to claim 13, wherein the co-solvent is ethanol and/or acetone and is present in an amount of 0.1% w/w to 80% w/w, respectively, of the aqueous solution.

15. A method according to claim 1, wherein the polymer is poly(methyl vinyl ether/maleic anhydride) (PMVE/MA) and is present in an amount of 0.5% w/w to 50% w/w of the aqueous solution.

16. A method according to claim 1, wherein the backing layer comprises a polyvinylchloride (PVC) emulsion and diethylphthalate.

17. A method according to claim 1, wherein the film formed in (c) is dried for a period of less than 30 minutes.

18. A method according to claim 1, wherein the step of drying the film comprises:
providing an air drier for drying the film, wherein an airflow venturi having a housing and a plurality of fans located within at least one wall thereof and adapted such that the fans can draw in warm air having a temperature of between 5° C. and 150° C.;
placing said film layer to be dried in the air drier; and
blowing the air over the film, said drier optionally containing within the housing a thermostatically controlled hot plate on which the film is placed.

19. A method according to claim 18, wherein the fan draws in warm air having a temperature in the range of 15° C. and 80° C.

20. A method according to claim 18, wherein the drier contains a thermostatically controlled hot plate and the hot plate is maintained at a temperature in the range of 15° C. to 100° C.

21. The method of claim 18, wherein said blowing warm air over the film layer is for a period of about 15 minutes or until the film layer is touch dry, whichever is shorter.

22. A method according to claim 1, wherein the step of drying the film comprises using infrared lamp(s) and/or microwave generator(s) to heat the film, whereupon or during which heating period cold air is optionally blown over the film.

23. A method of making a moist, layered bioadhesive patch, comprising:
(a) preparing a first monolayer film by:
(i) providing a first aqueous solution comprising at least one polymer selected from the group consisting of poly(methyl vinyl ether/maleic acid) and esters/amides thereof, poly(methyl vinyl ether/maleic anhydride) (PMVE/MA) and esters/amides thereof, and poly(acrylic acids) and esters/amides thereof; and at least one pharmaceutically active compound;
(ii) spreading out or spraying a thin layer of the first aqueous solution to form a first film;
(iii) drying the formed first film;
(iv) applying a layer of the first film on a backing;
(b) preparing a second monolayer film by:
(i) providing a second aqueous solution comprising at least one polymer selected from the group consisting of poly(methyl vinyl ether/maleic acid) and esters/amides thereof, poly(methyl vinyl ether/maleic anhydride) (PMVE/MA) and esters/amides thereof, and poly(acrylic acids) and esters/amides thereof; and at least one pharmaceutically active compound;
(ii) spreading out or spraying a thin layer of the second aqueous solution to form a second film;
(iii) drying the formed second film
(c) applying a layer of the second film on the first film layer;
(d) pressing the first film layer and second film layer together until the layers adhere.

24. A method according to claim 23, wherein the pharmaceutically active compound of the first monolayer film and the pharmaceutically active compound of second monolayer film are independently selected from the group consisting of nicotine, 5-ALA and derivatives thereof, antibiotics, parasympatholytics, cholinergics, neuroleptics, antidepressants, antihypertensives, photosensitisers, photosensitiser precursors, sympathomimetics, sympatholytics and antisympathotonics, antiolytics, local anaesthetics, central analgesics, antirheumatics, coronary therapeutics, hormones, antihistamines, prostaglandin derivatives, vitamins, nutrients and cytostatics.

25. A method according to claim 23, wherein the thickness of each film layer is from 1 μm to 500 μm.

26. A method according to claim 23, wherein the thickness of each film layer is from 25 μm to 75 μm.

27. A method according to claim 23, wherein each film layer exhibits a tensile strength greater than 1.0×10⁻⁸N cm⁻² and a residual tackiness, such that detachment of two layers of the same material requires a force of removal greater than 1.0 N cm⁻².

28. A method according to claim 23, wherein the first aqueous solution and/or the second aqueous solution further comprises at least one plasticizer independently chosen from among glycerol, propylene glycol, poly(ethylene glycol) and tripropylene glycol monomethyl ether (TPM).

29. A method according to claim 23, wherein the first aqueous solution and/or the second aqueous solution further comprises a water-miscible co-solvent.

30. A method according to claim 23, wherein the pharmaceutically active compounds 5-aminolevulinic acid (5-ALA) or a derivative or salt thereof and an analgesic are present in different layers of the bioadhesive patch.

31. A method according to claim 23, wherein the films formed are each in (iii) dried for a period of less than 30 minutes.

32. A method of making a moist, layered bioadhesive patch, comprising:
(a) preparing a first monolayer film by:
(i) providing a first aqueous solution comprising at least one polymer selected from the group consisting of poly(methyl vinyl ether/maleic acid) and esters/amides thereof, poly(methyl vinyl ether/maleic anhydride) (PMVE/MA) and esters/amides thereof, and poly(acrylic acids) and esters/amides thereof; and at least one pharmaceutically active compound;
(ii) spreading out or spraying a thin layer of the first aqueous solution to form a first film;
(iii) drying the formed first film;
(iv) applying a layer of the first film on a backing layer;
(b) preparing a second monolayer film by:
(i) providing a second aqueous solution comprising at least one polymer selected from the group consisting of poly(methyl vinyl ether/maleic acid) and esters/amides thereof, poly(methyl vinyl ether/maleic anhydride) (PMVE/MA) and esters/amides thereof, and poly(acrylic acids) and esters/amides thereof; and at least one pharmaceutically active compound;
(ii) spreading out or spraying a thin layer of the second aqueous solution to form a second film;
(iii) drying the formed second film;
(c) preparing an intermediate monolayer film by:
(i) providing a third aqueous solution comprising at least one polymer selected from the group consisting of poly(methyl vinyl ether/maleic acid) and esters/amides thereof, poly(methyl vinyl ether/maleic anhydride) (PMVE/MA) and esters/amides thereof, and poly(acrylic acids) and esters/amides thereof;
(ii) spreading out or spraying a thin layer of the third aqueous solution to form an intermediate film;
(iii) drying the formed intermediate film;
(d) applying the intermediate film layer onto the first film layer, on the side opposite the backing layer;
(e) pressing the intermediate film layer and first film layer together until the layers adhere;
(f) applying the second film layer onto the intermediate film layer, whereby the intermediate film layer is between the first and second film layers;
(g) pressing the second film layer and intermediate film layer together until the layers adhere.

33. A method according to claim 32, wherein the pharmaceutically active compound of the first monolayer film and the pharmaceutically active compound of second monolayer film are independently selected from the group consisting of nicotine, 5-ALA and derivatives thereof, antibiotics, parasympatholytics, cholinergics, neuroleptics, antidepressants, antihypertensives, photosensitisers, photosensitiser precursors, sympathomimetics, sympatholytics and antisympathotonics, antiolytics, local anaesthetics, central analgesics, antirheumatics, coronary therapeutics, hormones, antihistamines, prostaglandin derivatives, vitamins, nutrients and cytostatics.

34. A method according to claim 32, wherein the thickness of each film layer is from 1 μm to 500 μm.

35. A method according to claim 32, wherein the thickness of each film layer is from 25 μm to 75 μm.

36. A method according to claim 32, wherein each film layer exhibits a tensile strength greater than 1.0×10⁻⁸N cm⁻² and a residual tackiness, such that detachment of two layers of the same material requires a force of removal greater than 1.0 N cm⁻².

37. A method according to claim 32, wherein the first aqueous solution and/or the second aqueous solution and/or the third solution further comprises at least one plasticizer independently chosen from among glycerol, propylene glycol, poly(ethylene glycol) and tripropylene glycol monomethyl ether (TPM).

38. A method according to claim 32, wherein the first aqueous solution and/or the second aqueous solution and/or the third solution further comprises a water-miscible co-solvent.

39. A method according to claim 32, wherein the pharmaceutically active compounds 5-aminolevulinic acid (5-ALA) or a derivative or salt thereof and an analgesic are present in different layers of the bioadhesive patch.

40. A method according to claim 32, wherein the films formed are each in (iii) dried for a period of less than 30 minutes.

41. A method according to claim 32, wherein at least one of the aqueous solutions comprises tripropylene glycol monomethyl ether (TPM) plasticizer and is present in an amount in the range of 0.25 to 25% w/w of the at least one aqueous solution.

42. A method according to claim 32, wherein at least one of the aqueous solutions comprises ethanol and/or acetone co-solvent and is present in an amount of 0.1% w/w to 80% w/w, respectively, of the at least one aqueous solution.
